(19) United States
(12) Patent Application Publication
(10) Pub. No.: US 2014/0025031 A1
(43) Pub. Date: Jan. 23, 2014

(54) FLEXIBLE, SELF EXPANSIBLE, REMOVABLE MEMORY COIL INTRAGASTRIC DEVICE AND METHOD OF USING SUCH DEVICE FOR WEIGHT REDUCTION AND MEDICATION DELIVERY

(71) Applicant: Chunwang LAM, Easton, CT (US)

(72) Inventor: Chunwang LAM, Easton, CT (US)

(21) Appl. No.: 13/555,357

(22) Filed: Jul. 23, 2012

(51) Int. Cl. A61M 29/00; A61M 31/00

(52) U.S. Cl. 604/500; 606/191

(57) ABSTRACT

An exemplary embodiment of the present invention is directed to a mechanical device that is intended to be used as an adjunct or alternative to diet, exercise or medication to help individual lose weight. The mechanical device may be an intragastric device that is self expansible, reusable, removable coil delivered endoscopically and deployed internally in the human stomach. When deployed in the human stomach, the intragastric device is configured to cause a state of constant satiety, thereby helping patients who have a moderate obesity problem lose weight. Another exemplary embodiment of the invention is intended for use in delivering pharmaceutical agents in a continuous, steady state, and reliable fashion over an extended period of time using the stomach as the natural reservoir.

### BACKGROUND OF THE INVENTION

1. Technical Field

The present invention relates generally to intragastric devices used for weight reduction and/or medication delivery. More particularly, the present invention relates to a flexible, self expansible removable intragastric coil device that may be delivered endoscopically and deployed internally in the stomach.

2. Description of the Background Art

U.S. Appl. Publ. No. 2007/0239284 is directed to an obesity treatment apparatus that includes an intragastric member comprising a curvilinear axis which extends about and along a central axis of an intragastric device. The curvilinear axis of the intragastric member is spaced away from the central axis by a predetermined distance or a variable distance. The intragastric member comprises a shape selected from one of a spiral, helix, coil, cork screw, spring and loop. The obesity treatment apparatus may further include an intragastric member including a proximal end, a distal end and a lumen extending between the proximal end and the distal end, and the lumen may be utilized to inflate the intragastric member to the second configuration. The intragastric member may also include a self-expanding metal, such as nitinol. The intragastric member is delivered by mounting the intragastric member over a plastic overtube compressed by a sheath and secured by retaining elements. In order to deliver the intragastric member the retaining elements and sheath must be removed, which can add to the complexity and difficulty of delivering the intragastric member.

U.S. Appl. Publ. No. 2008/0249635 is directed to a method of treating obesity that includes inserting to a desired position within the GI tract a filament filler in a substantially straight configuration and moving the filler into an operative configuration in which the filler curves along a predetermined path to define a desired volume in combination with, after a predetermined treatment period has elapsed, collapsing the filler into the substantially straight configuration for trans-oral removal. The filament filler is intended to fill up a certain volume of the GI tract in order to limit the amount of caloric intake that is desired.

U.S. Appl. No. 2008/0161935 is directed to a method for inducing weight loss in a patient by providing an implant for placement within the stomach. The implant has a member with an undeployed shape for delivery to the stomach and a deployed shape for implantation therein. The member has a plurality of links pivotally connected to each other, and a flexible elongated tether connected to the member. The method then involves delivering the member to the stomach while in its undeployed shape, and placing the member in its deployed shape by applying tension to the tether.

U.S. Pat. No. 8,070,768 is directed to an implantable device that includes an embodiment of a mechanically expandable device that includes expandable coils routed through loops that are fixed to membrane. The non-expanded shape of membrane may function to guide the overall expanded shape of device. Membrane can be made from a compliant material, such as an elastomer, or from a semi-compliant or noncompliant material, or from some combination of these three classes of materials. Membrane can include reinforcing structure at the locations where loops are affixed thereto. After device is positioned where it is desired to implant the device, a wire is advanced into membrane to expand the diameter of coils, thereby expanding the device into its expanded conformation.

U.S. Pat. No. 5,868,141 is directed to an endoscopic stomach insert for treating obesity in humans by reducing the desire for eating, comprising a base-sized for passing through a human mouth and esophagus; a plurality of flexible blades coupled at one end thereof to the base and circumferentially arranged about the base central axis, where the blades are biased to extend substantially radially outward and downward from the base; and a retainer for releasably coupling the distal portions of the blades within close proximity to each other about the central axis of the base. The insert is thus adapted to be passed through the mouth and esophagus and into a human stomach, and upon releasing the retainer within the stomach, the blades are biased to flare outwardly into the form of a dome-shaped cage, applying pressure to the stomach, and thus causing a sensation of fullness within the stomach and reducing the desire for eating.

U.S. Appl. Publ. No. 2007/0225564 is directed to a medical device, for example, an endoscope or catheter, that includes local drug delivery capabilities for selectively delivering at least one drug in vivo. The local drug delivery may occur as the medical device is advanced through tortuous passageways of the patient's body or may occur after the medical device has reached its targeted destination. The medical device includes a drug agent, for example, carried in or on a hydrophilic or hydrogel coating disposed on the outside thereof. When the hydrogel or drug agent receives an appropriate signal, e.g., solution containing a triggering agent or triggering condition, e.g., heat or light, the hydrogel contracts or expands to squeeze out the drug from hydrogel.

U.S. Appl. Publ. No. 2009/0104243 is directed to a solid drug core insert that can be manufactured by injecting a liquid mixture comprising a therapeutic agent and a matrix precursor into a sheath body. The mixture is cured to form a solid drug-matrix core. A surface of the solid drug core is exposed, for example by cutting the tube, and the exposed surface of the solid drug core releases therapeutic quantities of the therapeutic agent when implanted into the patient.

However, conventional intragastric devices attempt to treat obesity by taking up volume within the stomach of a patient. These types of devices are prone to potential problems such as migration, deflation and gastric ischemia. Furthermore, there does not appear to be adequate mechanisms for the delivery of drugs and other pharmaceutical agents over an extended period of time from within the digestive tract.

### SUMMARY OF THE INVENTION

The present invention is designed to overcome the above noted limitations that are attendant upon the use of conventional intragastric and drug delivery devices and, toward this end, it contemplates the provision of a novel intragastric device that is configured for use in the treatment of obesity and/or delivery of medication through the gastrointestinal tract.

It is an object of the present invention to provide a mechanical device that is intended to be used as an adjunct or alternative to diet, exercise or medication to help individual lose weight.

It is another object of the present invention to provide a mechanical device that may be used to deliver pharmaceutical agents in a continuous, steady state, and reliable fashion over an extended period of time using the stomach as the natural reservoir.

It is yet another object of the present invention to provide for internal medication administration by storing a large amount of medication in the stomach to be dissolved slowly over the course of weeks to months to achieve a reliable means of providing medicinal treatment for chronic medical conditions.

It is still another object of the present invention to provide a flexible coil configured to gently expand the stomach, causing a sense of fullness, thereby limiting an individual's desire to eat.

It is yet another object of the present invention that the method of deployment of the mechanical device according to the present invention is minimally invasive and readily adaptable to the current standard endoscopic practice.

It is still another object of the present invention to provide an intragastric device that eliminates potential problems of migration, deflation and gastric ischemia.

It is yet another object of the present invention that the method of medication delivery according to the present invention has minimal modification of the currently available pharmaceutical agents and increases patient compliance.

It has now been found that the foregoing and related objects can be readily attained in a self expansible, reusable, removable intragastric device delivered endoscopically and deployed internally in the human stomach, and a method for deploying the same.

Accordingly, an exemplary embodiment of the present invention is directed to an intragastric device that includes a strand having a first end and a second end, the strand is configurable between a substantially linear configuration of the intragastric device and a substantially coiled configuration of the intragastric device.

In accordance with the exemplary embodiment of the present invention, when in the substantially coiled configuration of the intragastric device, the strand is coiled so as to produce a plurality of spaced apart loops.

In accordance with the exemplary embodiment of the present invention, the plurality of spaced apart loops of the strand are positioned to define an interior region of the substantially coiled configuration of the intragastric device.

In accordance with the exemplary embodiment of the present invention, the substantially coiled configuration of the intragastric device is at least partially compressible in a first direction.

In accordance with the exemplary embodiment of the present invention, the strand is configured to resiliently return to the substantially coiled configuration of the intragastric device when placed in the substantially linear configuration of the intragastric device.

In accordance with the exemplary embodiment of the present invention, when in the substantially coiled configuration of the intragastric device, the first end and the second end are each oriented so as to extend into the interior region of the substantially coiled configuration of the intragastric device.

In accordance with the exemplary embodiment of the present invention, a shape of the substantially coiled configuration of the intragastric device defined by the plurality of spaced apart loops is one of spheroid, ovoid, ellipsoid, stomach or bean.

In accordance with the exemplary embodiment of the present invention, the intragastric device is configured in the substantially coiled configuration of the intragastric device to expand a stomach of a patient in order to produce a sense of fullness in the patient.

In accordance with the exemplary embodiment of the present invention, the strand includes a longitudinal axis, and at least a portion of the strand is made of a helical coil positioned around the longitudinal axis of the strand, and the helical coil defines a bore within the portion of the strand comprised of the helical coil.

In accordance with the exemplary embodiment of the present invention, the bore of the helical coil includes a cylindrical medication core disposed in at least a portion of the bore.

In accordance with the exemplary embodiment of the present invention, the strand is made of a resilient material having shape memory.

In accordance with the exemplary embodiment of the present invention, the resilient material includes titanium and/or a titanium alloy.

According to another exemplary embodiment of the present invention, a method of treating obesity using an intragastric device is provided, and the method includes implanting the intragastric device into a gastrointestinal tract of a patient.

In accordance with this other exemplary embodiment of the present invention, the intragastric device includes a strand having a first end and a second end, the strand is configurable between a substantially linear configuration of the intragastric device and a substantially coiled configuration of the intragastric device, in the substantially coiled configuration of the intragastric device the strand is coiled so as to produce a plurality of spaced apart loops, the plurality of spaced apart loops of the strand are positioned to define an interior region of the substantially coiled configuration of the intragastric device, and the substantially coiled configuration of the intragastric device is at least partially compressible in a first direction.

In accordance with this other exemplary embodiment of the present invention, the intragastric device is configured in the substantially coiled configuration of the intragastric device to expand a stomach of the gastrointestinal tract of the patient in order to produce a sense of fullness in the patient.

In accordance with this other exemplary embodiment of the present invention, implanting the intragastric device into the gastrointestinal tract of the patient includes configuring the strand into the substantially linear configuration of the intragastric device, inserting the first end of the strand into an opening of the gastrointestinal tract of the patient, moving the first end of the strand along the gastrointestinal tract of the patient towards a stomach of the gastrointestinal tract of the patient, and inserting the second end of the strand into the stomach of the gastrointestinal tract of the patent so that the strand resiliently returns to the substantially coiled configuration of the intragastric device within the stomach.

In accordance with this other exemplary embodiment of the present invention, the strand includes a longitudinal axis, and at least a portion of the strand is made of a helical coil positioned around the longitudinal axis of the strand, and the helical coil defines a bore within the portion of the strand made of the helical coil.

In accordance with this other exemplary embodiment of the present invention, the strand is made of a resilient material having shape memory.

In accordance with this other exemplary embodiment of the present invention, the resilient material includes titanium and/or a titanium alloy.

According to another exemplary embodiment of the present invention, a method of administering at least one medication to a patient using an intragastric device is provided, and the method includes implanting the intragastric device into a gastrointestinal tract of a patient.

In accordance with this other exemplary embodiment of the present invention, the intragastric device includes a strand having a first end and a second end, the strand is configurable between a substantially linear configuration of the intragastric device and a substantially coiled configuration of the intragastric device, in the substantially coiled configuration of the intragastric device the strand is coiled so as to produce a plurality of spaced apart loops, the plurality of spaced apart loops of the strand are positioned to define an interior region of the substantially coiled configuration of the intragastric device, and the substantially coiled configuration of the intragastric device is at least partially compressible in a first direction.

In accordance with this other exemplary embodiment of the present invention, the strand includes a longitudinal axis, and at least a portion of the strand is made of a helical coil positioned around the longitudinal axis of the strand, and the helical coil defines a bore within the portion of the strand made of the helical coil.

In accordance with this other exemplary embodiment of the present invention, the bore of the helical coil includes a cylindrical medication core disposed in at least a portion of the bore.

In accordance with this other exemplary embodiment of the present invention, implanting the intragastric device into the gastrointestinal tract of the patient includes configuring the strand into the substantially linear configuration of the intragastric device, inserting the first end of the strand into an opening of the gastrointestinal tract of the patient, moving the first end of the strand along the gastrointestinal tract of the patient towards a stomach of the gastrointestinal tract of the patient, and inserting the second end of the strand into the stomach of the gastrointestinal tract of the patent so that the strand resiliently returns to the substantially coiled configuration of the intragastric device within the stomach.

In accordance with this other exemplary embodiment of the present invention, the cylindrical medication core includes the at least one medication, and is configured to release the at least one medication into the gastrointestinal tract of the patient once the intragastric device has been implanted into the gastrointestinal tract of the patient.

In accordance with this other exemplary embodiment of the present invention, the strand is made of a resilient material having shape memory.

In accordance with this other exemplary embodiment of the present invention, the resilient material includes titanium and/or a titanium alloy.

### BRIEF DESCRIPTION OF THE DRAWINGS

Having thus described the invention in general terms, reference will now be made to the accompanying drawings, which are not necessarily drawn to scale, where:

FIG. 1 is a side view of an exemplary embodiment of an intragastric device in accordance with the present invention;

FIG. 2 is a close-up detail view of an end portion of the exemplary intragastric device from FIG. 1;

FIG. 2A is a close-up and partially cut-away view of the end portion from FIG. 2 of the exemplary intragastric device;

FIG. 3 is a schematic view showing an exemplary procedure for implantation of the exemplary intragastric device into a patient;

FIG. 4 is a schematic view showing an exemplary intragastric device implanted into a patient and in use;

FIG. 5 is a side view of another exemplary embodiment of an intragastric device in accordance with the present invention;

FIG. 6 is a close-up detail view of another exemplary end portion of the intragastric device in accordance with the present invention; and

FIG. 6A is a cross-section view of the end portion of the exemplary intragastric device taken along line 6A-6A in FIG. 6.

### DETAILED DESCRIPTION OF THE INVENTION

The present invention now will be described more fully hereinafter with reference to the accompanying figures, in which exemplary embodiments of the invention are shown. The invention may, however, be embodied in many different forms and should not be construed as limited to the embodiments set forth herein. Like reference numerals refer to like elements throughout.

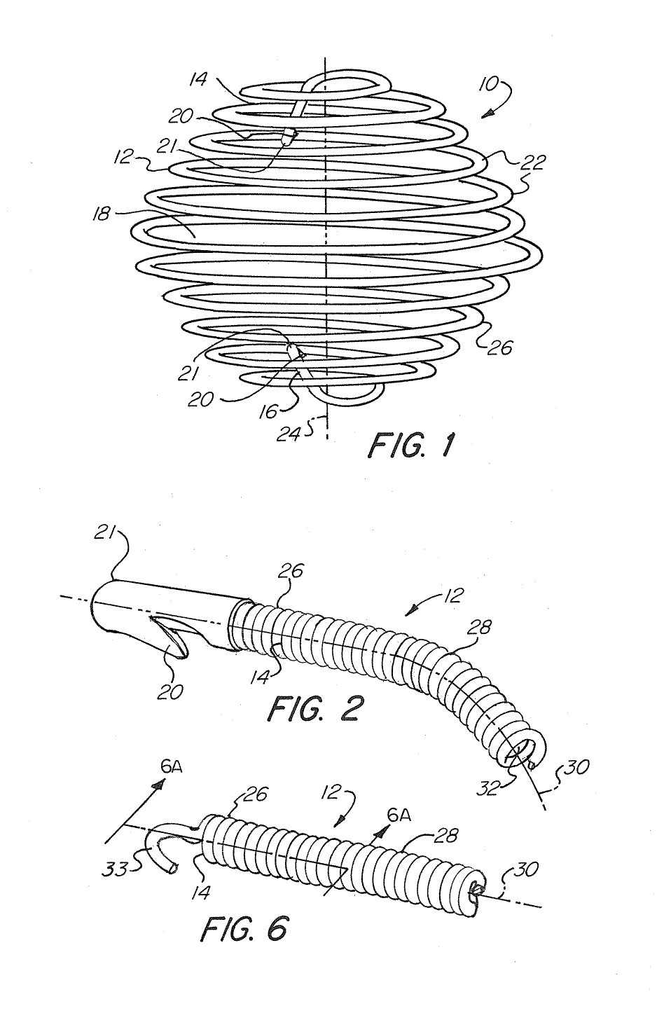

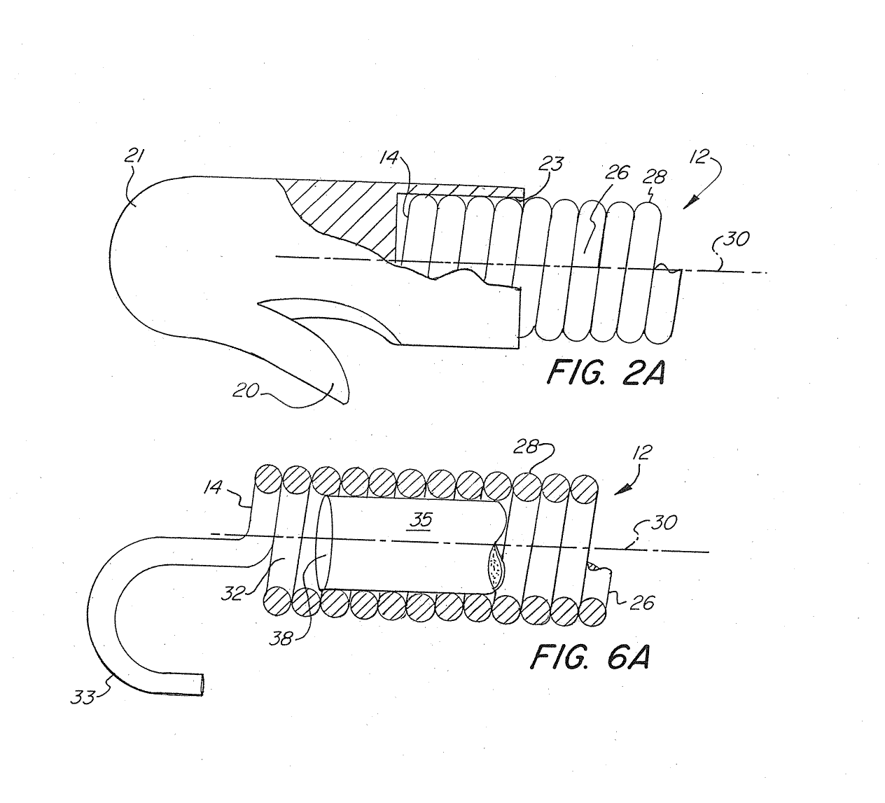

FIGS. 1-2, 2A and 3-4 show an exemplary embodiment of an intragastric device, generally indicated by reference numeral 10, according to the present invention. The intragastric device 10 may be formed from a gastric coil 12. The gastric coil 12 includes a first end 14 and a second end 16, which are positioned at the ends of the gastric coil 12 and may be oriented so that the first end 14 and the second end 16 are pointing in a direction towards the interior region 18 of the gastric coil 12. In this manner, the first end 14 and the second end 16 are disposed within the interior region 18 of the gastric coil 12 so that the gastric coil 12 does not have any protrusions extending therefrom. As shown in greater detail in FIG. 2, each of the first end 14 and the second end 16 may include a hook-like configuration 20 disposed thereon. While only the first end 14 is shown for simplicity in FIG. 2, it is understood that second end 16 may also be configured in a like manner. The hook-like configuration 20 is configured to assist in the removal of the intragastric device 10 from a patient whom has had the intragastric device 10 implanted into the digestive tract of the patient. As shown for example in FIG. 2, the hook-like configuration 20 may be positioned on, and extending from an end cap 21 that has been engaged with and secured to the first end 14 of the gastric coil 12. The end cap 21 positioned over the first end 14 of the gastric coil 12. As shown in greater detail in FIG. 2A, the end cap 21 includes a hollow interior cavity 23, and is dimensioned so that the end cap 21 can securely engage the first end 14 and slide up over at least a portion of the gastric coil 12. The hook-like configuration 20 can extend from any portion of the end cap 21. While the end cap 21 has been discussed with respect to the first end 14 of the gastric coil 12, it is understood that the end cap 21 may be positioned on either or both the first end 14 and the second end 16 of the gastric coil 12.

Referring again to FIG. 1, the gastric coil 12 is formed from one or more loops 22 that are positioned in a spaced apart relationship substantially concentric with each other. The one or more loops 22 are oriented substantially perpendicular to the longitudinal axis 24 of the gastric coil 12. It is understood that the one or more loops 22 of the gastric coil 12 are formed from a single strand 26 of material so that the gastric coil 12 is a continuous strand of material. However, it is also understood that the gastric coil 12 according to the present invention may also include gastric coils 12 formed from multiple strands (not shown) of materials joined together either prior to implantation into the patient or subsequent to insertion into the patient. It is further understood that the strand 26 used to form the gastric coil 12 may have any cross-sectional shape, for example circular, oval or elliptical. It may be preferable that the strand 26 has a cross-sectional shape with a rounded surface and/or surfaces in order reduce the likelihood of the gastric coil 12 causing damage to the patient.

Referring to FIGS. 2 and 2A, the strand 26 that forms the gastric coil 12 may itself be made from a smaller coiled strand 28 that is coiled about a longitudinal axis 30 of the strand 26. The smaller coiled strand 28 is coiled so that an interior cavity 32 is formed within the strand 26. It is understood that in accordance with the present invention the entire length of the strand 26 may be formed from the smaller coiled strand 28, only a section of the length of the strand 26 may be formed from the smaller coiled strand 28 or multiple sections of the length of the strand 26 may be formed from multiple smaller coiled strands 28. For example, only the first end 14 or the second end 16 of the gastric coil 12 may be formed from the smaller coiled strand 28, and the remaining length of the strand 26 of the gastric coil 12 may be formed from a solid strand.

Referring now to FIGS. 6 and 6A, the first end 14 of the gastric coil 12 is shown with another exemplary hook-like configuration 33. In this exemplary configuration, the hook-like configuration 33 is formed integrally from the smaller coiled strand 28, so that no additional caps or other hooks are needed to be included on the gastric coil 12. The hook-like configuration 33 shown in FIGS. 6 and 6A is dimensioned, oriented and configured in an appropriate manner in order to assist in the removal of the intragastric device 10 from a patient whom has had the intragastric device 10 implanted into the digestive tract of the patient. Therefore, it is understood that the exemplary embodiment of the intragastric device 10 shown in FIG. 1 may include either the hook-like configuration 20 shown in FIGS. 2 and 2A and/or the hook-like configuration 33 shown in FIGS. 6 and 6A. It is understood that while FIGS. 6 and 6A show the first end 14 of the gastric coil 12, the hook-like configuration 33 may also be formed on the second end 16 of the gastric coil 12. Furthermore, it is possible that the gastric coil 12 may include the hook-like configuration 20 from FIGS. 2 and 2A on the first end 14 and the hook-like configuration 33 from FIGS. 6 and 6A on the second end 16, or any other combination of hook-like configurations 20, 33.

Referring now to FIG. 6A, it is understood that the interior cavity 32 of the strand 26 may be left empty or a substance, such as a substantially cylindrical medication core 35, may be included in the interior cavity 32. The medication core 35 may be wrapped in an impermeable or semi-impermeable membrane (not shown) so that only one face 38 of the medication core 35 is exposed. In this manner, the medication core 35 can be configured to provide controlled release of the medication once the gastric coil 12 has been implanted into the patient. It is also understood that the medication core 35 may not be wrapped in the membrane (not shown), and designed so that the medication core 35 provides controlled release of the medication from all surfaces of the medication core 35. It is also understood that the medication within the medication core 35 may be infused with an undissolvable or substantially undissolvable substance (not shown) to form the medication core 35 so that as the medication is dissolved and released the medication core 35 maintains its position within the interior cavity 32 of the strand 26 so that the medication core 35 is not prematurely expelled from the gastric coil 12 as a result of shrinkage of the medication core 35 due to the release of the medication contained within the medication core 35.

While the medication core 35 is only shown in FIG. 6A with respect to the exemplary embodiment of the gastric coil 12 with the hook-like configuration 33 of FIG. 6A, it is understood that a medication core may also be included in the exemplary embodiment of the gastric coil 12 with the end cap 21 shown in FIG. 2A. Accordingly, it is further understood that the medication from the medication core in such an embodiment of the present invention may be released from the spaces between the coils of the smaller coiled strand 28, and that the medication core may be positioned within the interior cavity 32 so that the end cap 21 does not interfere with the release of the medication from the medication core. It is also understood that the end cap 21 may be perforated (not shown) in order to allow and/or aid in the release of medication from the medication core in the exemplary embodiment of the gastric coil 12 shown in FIGS. 2 and 2A.

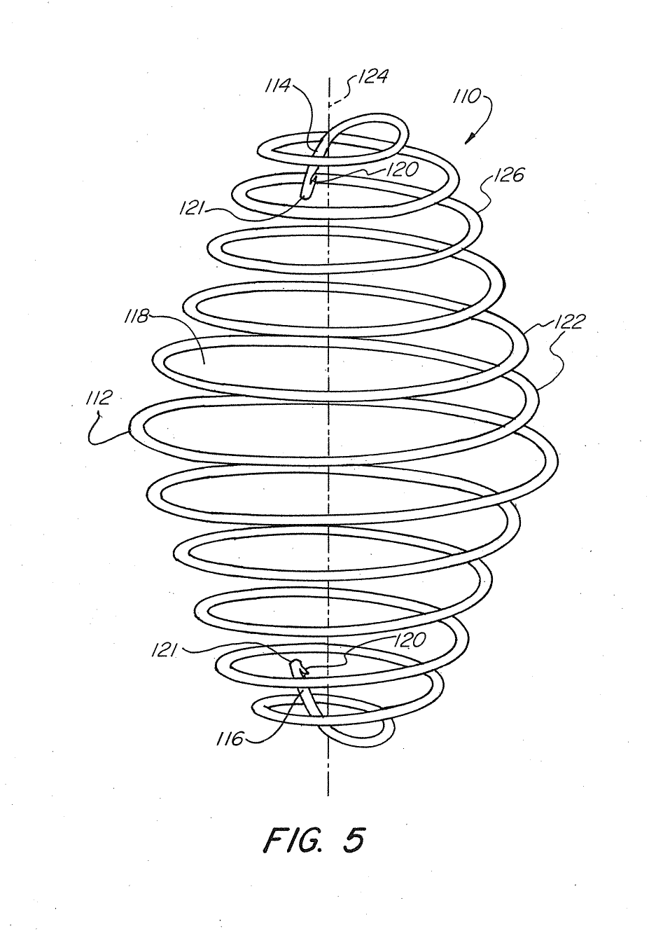

The gastric coil 12 may be formed from any suitable memory metal, for example a titanium alloy such as nickel titanium, or a durable plastic. It is preferable that the gastric coil 12 be formed from inert materials, and more preferable that the gastric coil 12 is formed from materials that do not readily react with the environments in which the gastric coil 12 is intended to be deployed, for example the stomach of the patient. The gastric coil 12 is configured to be self expansible after being delivered and implanted into the stomach of the patient. The gastric coil 12 may also be removable and reusable once removed from the stomach of the patient as will be discussed further below with respect to the operation and use of the gastric coil 12. The gastric coil 12 may be shaped in a variety of forms as defined by the one or more loops 22 of the stand 26 forming the gastric coil 12. For example, the gastric coil 12 may be in the shape of a spheroid as shown for example in FIG. 1. However, it is understood that the gastric coil 12 may also be formed in the shape of a prolate spheroid, oblate spheroid, ovoid, bean, stomach or ellipsoid, as shown for example by the gastric coil 112 in FIG. 5.

Referring now to FIG. 5, an alternative embodiment of an intragastric device, generally indicated by reference numeral 110, according to the present invention is shown. The intragastric device 110 may be formed from the gastric coil 112. The gastric coil 112 includes a first end 114 and a second end 116, which are positioned at the ends of the gastric coil 112 and may be oriented so that the first end 114 and the second end 116 are pointing in a direction towards the interior region 118 of the gastric coil 112. In this manner, the first end 114 and the second end 116 are disposed within the interior region 118 of the gastric coil 112 so that the gastric coil 112 does not have any protrusions extending therefrom. Each of the first end 114 and the second end 116 may include a hook-like configuration 120 extending therefrom. The hook-like configuration 120 is configured to assist in the removal of the intragastric device 110 from a patient whom has had the intragastric device 110 implanted into the digestive tract of the patient. The hook-like configuration 120 may be positioned on, and extending from an end cap 121 that has been engaged with and secured to the first end 114 and/or the second end 116 of the gastric coil 112.

Still referring to FIG. 5, the gastric coil 112 is formed from one or more loops 122 that are positioned in a spaced apart relationship substantially concentric with each other. The one or more loops 122 are oriented substantially perpendicular to the longitudinal axis 124 of the gastric coil 112. It is understood that the one or more loops 122 of the gastric coil 112 are formed from a single strand 126 of material so that the gastric coil 112 is a continuous strand of material. However, it is also understood that the gastric coil 112 according to the present invention may also include gastric coils 112 formed from multiple strands (not shown) of materials joined together either prior to implantation into the patient or subsequent to insertion into the patient. It is further understood that the strand 126 used to form the gastric coil 112 may have any cross-sectional shape, for example circular, oval or elliptical. It may be preferable that the strand 126 has a cross-sectional shape with a rounded surface and/or surfaces in order reduce the likelihood of the gastric coil 112 causing damage to the patient.

Referring again the FIG. 1, it is understood that the gastric coil 12 so as to have the particular shape and configuration as shown for example in FIG. 1. However, it is also understood that the gastric coil 12 is pliable in a variety of directions as a result of the materials that maybe used to form the gastric coil 12 and the configuration of the strand 26 that forms the gastric coil 12. For example, it is understood that the gastric coil 12 maybe substantially compressed or extended in a direction substantially perpendicular to the longitudinal axis 24 of the gastric coil 12. Furthermore, it is also understood that the one or more loops 22 may be urged in a direction substantially perpendicular to the longitudinal axis 24 of the gastric coil 12 and/or the one or more loops 22 may be compressed toward one another on one side of the gastric coil 12 and extended away from one another on an opposing side of the gastric coil 12. It is understood that the pliability of the gastric coil 12 permits its efficient use as an intragastric device 10 in order to reduce the discomfort that may be felt by the patient whom has had the gastric coil 12 implanted into the patient's stomach.

Still referring to FIG. 1, it is understood that the length of the strand 26 used to form the gastric coil 12 may be any suitable length and may vary depending upon the intended patient. Preferably, the strand 26 used to form the gastric coil 12 may be approximately one meter in length. Similarly, it is understood that the diameter of the strand 26 may be any suitable length and may vary depending upon the intended application for the gastric coil 12 and/or the intended patient for the gastric coil. Preferably, the strand 26 may be approximately 2.5 to 3.0 mm, and more preferably 2.8 mm. It is further understood that the diameter of the gastric coil 12 will vary depending upon the shape formed by the gastric coil 12, and also depend upon the intended application for the gastric coil 12 and/or the intended patient for the gastric coil. For example, with respect to the gastric coil 12 shown in FIG. 1 that has a substantially spheroid shape, the diameter of the gastric coil 12 may be from approximately 8 to 16 cm. However, it is understood that the gastric coil 12 may have any suitable diameter and/or diameters. While the characteristics and dimensions of the gastric coil 12 shown in FIG. 1 have been discussed above, it is understood that the discussion of the characteristics and dimensions of the gastric coil 12 of FIG. 1 apply equally well to the gastric coil 112 shown in FIG. 5 as another exemplary embodiment of the present invention.

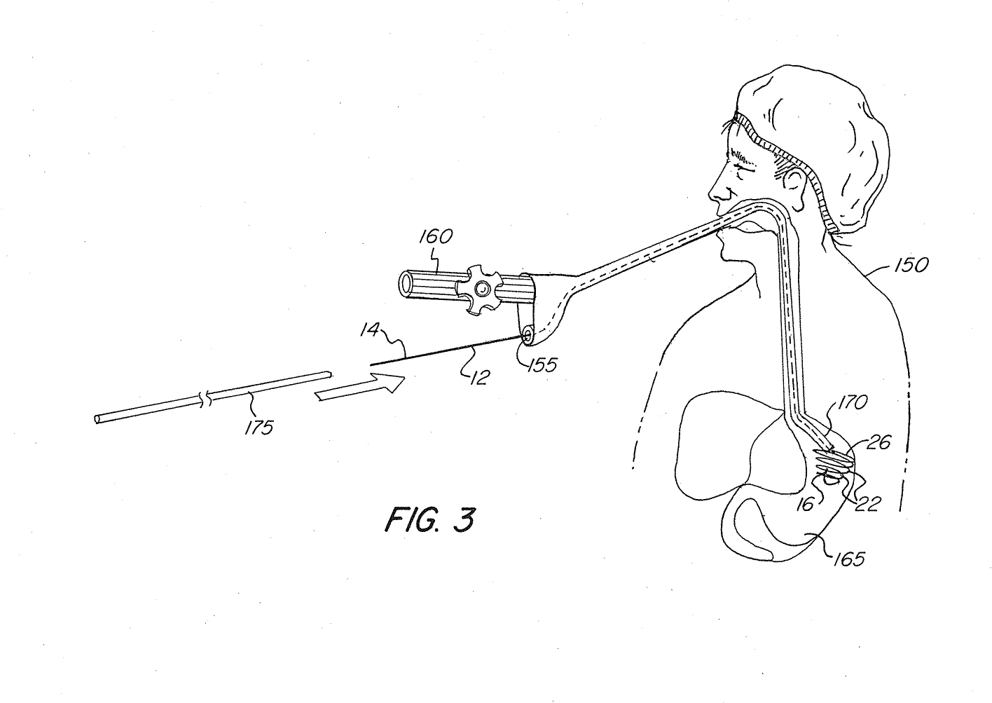

An exemplary method of implanting the gastric coil 12 into a patient and exemplary methods of using the gastric coil 12 will now be discussed in further detail by referring to FIG. 3. Once the intended patient 150 has signed an informed consent, the patient 150 is appropriately sedated, for example by the use of conscious sedation, by a licensed medical facility (not shown). The purpose of the sedation is to optimize the comfort of the procedure for the patient 150. After the patient 150 is sedated standard endoscopy will be performed by a licensed gastroenterologist to check for anatomic abnormalities, and the gastric coil 12 may also be checked for mechanical defects or other abnormalities that may interfere with the implantation procedure and/or the functionality of the gastric coil 12 once implanted into the patient 150. The first end 14 and the second end 16 of the gastric coil 12 are then pulled in a direction substantially away from each other, for example in opposite directions along the longitudinal axis 24 of the gastric coil 12 in order to substantially straighten the gastric coil 12 so that it has a substantially linear configuration.

Once the gastric coil 12 has been substantially straightened, either the first end 14 or the second end 16 is inserted into a working channel 155 of a video endoscope 160. The gastric coil 12 is then pushed along the length of the working channel 155 so that it begins to enter the stomach 165 of the patient 150. Once the gastric coil 12 begins to enter the stomach 165 and proceeds out the exit hole 170 of the working channel 155 the gastric coil 12 is no longer retained in the substantially straight configuration by the working channel 155, and begins to regain its preconfigured shape. For example, as shown in FIG. 3 the loops 22 of the gastric coil 12 begin to form as the strand 26 of the gastric coil 12 is inserted into the substantially empty space of the stomach 165 of the patient 150. After the entire length of the gastric coil 12 has been pushed into the working channel 155, a pusher catheter 175 may be used to complete the delivery of the entire length of the gastric coil 12 into the stomach 165 of the patient 150. The pusher catheter 175 should be removed prior to the removal of the video endoscope 160. The pusher catheter 175 may be reused after standard sterilization and/or disinfection procedures are performed. The placement of the gastric coil 12 in the stomach 165 can be visible via the video endoscope 160, and should be ascertained before the endoscope 160 is to be removed from the patient 150 in order to ensure proper and appropriate position and orientation of the gastric coil 12 within the stomach 165 of the patient 150.

In order to remove the gastric coil 12 from the stomach 165 of the patient 150 the video endoscope 160 may be reinserted into the patient 150 so that the working channel 155 is extended into the stomach 165 of the patient 150. Once the gastric coil 12 is located by the video endoscope 160, a standard endoscopic snare (not shown) may be inserted through the working channel 155 in order to grab and/or attach to either the first end 14 or the second end 16 of the gastric coil 12. The hook-like configuration 20 on each of the first end 14 and the second end 16 may facilitate the endoscopic snare (not shown) in grabbing and/or attaching to each end. After the gastric coil 12 is securely held by the endoscopic snare (not shown), the endoscopic snare may then be drawn back out the working channel 155 of the video endoscope 160 so that either the first end 14 or the second end 16 of the gastric coil 12 enter the exit hole 170 of the working channel 155. Due to the size of the working channel 155 and/or the exit hole 170 the gastric coil 12 is forced into a substantially linear configuration in order to allow removal of the gastric coil 12 from the patient 150. Once removed from the patient 150 the gastric coil 12 may be reused after standard sterilization and/or disinfection procedures are performed. While the implantation and removal of the gastric coil 12 shown in FIG. 1 have been discussed above, it is understood that the discussion of the implantation and removal of the gastric coil 12 of FIG. 1 apply equally well to the gastric coil 112 shown in FIG. 5 as another exemplary embodiment of the present invention.

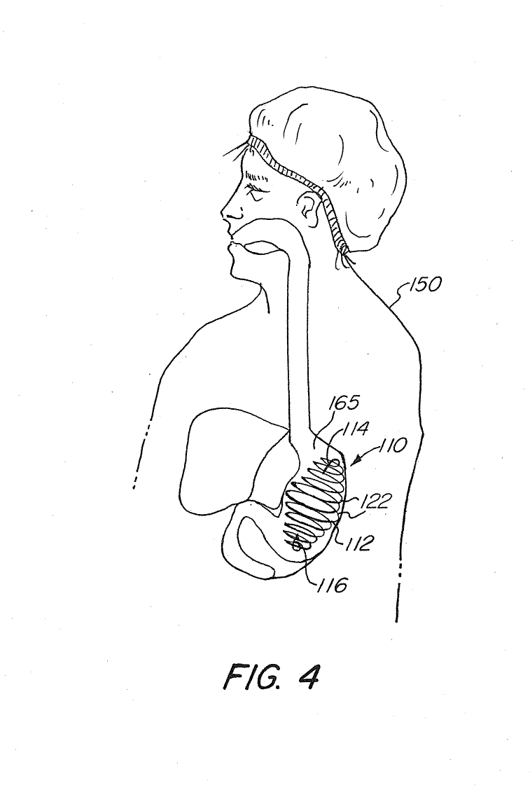

For example, referring now to FIG. 4, the gastric coil 112 shown in FIG. 5 is shown implanted into the stomach 165 of the patient 150. As shown in FIG. 4, the gastric coil 112 has regained its substantially ellipsoid configuration, and the one or more loops 122 of the gastric coil 112 have been reformed once the implantation procedure is completed.

Once implanted into the patient 150 the gastric coil 12 may be used for a variety of purposes. One such exemplary purpose is that the gastric coil 12 may be used as an adjunct or alternative to diet, exercise or medication to help individual lose weight. When deployed in the stomach 165, the gastric coil 12 may be configured to cause a state of constant satiety in the patient 150, thereby helping the patient 150 who has a moderate obesity problem lose weight. The gastric coil 12 may be used in this manner because the gastric coil 12 reforms into the shape of stomach 165 once implanted into the stomach 165 of the patient 150. When in the stomach 165, the tensile force of the gastric coil 12 will cause it to reform to its originally designed shape, thereby expanding the stomach 165, and causing a sense of fullness (satiety) at all times. Satiety is a subjective feeling rendered by a relatively expanded stomach, either filled with food or with inert matter such as air or water. The gastric coil 12 is flexible and therefore can be used to gently expand the stomach, causing a sense of fullness and thereby limiting the patient's 150 desire to eat. However, since the one or more loops 22 of the gastric coil 12 are spaced apart, and the gastric coil 12 is configured to have an interior region 18, particles, such as food, within the stomach 165 are free to pass through the gastric coil 12. As a result, the gastric coil 12 provides the benefit of gently expanding the stomach 165 to provide a sense of satiety without taking up a signification portion of the volume of the stomach 165. In this manner, certain negative side effects of other intragastric devices that are used for the purposes of weight loss can be avoided. For example, since the gastric coil 12 does not occupy a significant portion of the volume of the stomach 165 the gastric coil 12 does not substantially interfere with the normal digestive processes performed by the stomach 165.

Another exemplary purpose for which the gastric coil 12 according to the present invention may be used for is as a medication delivery mechanism. In this manner, the gastric coil 12 may be used to deliver pharmaceutical agents in a continuous, steady state, and reliable fashion over an extended period of time using the stomach 165 as a natural reservoir. According to one exemplary embodiment of the present invention the stomach 165 is used as a natural reservoir for internal medication administration, by storing a large amount of medication retained within the gastric coil 12 in the stomach 165 to be dissolved slowly over the course of a predetermined period of time, for example weeks to months, in order to achieve a reliable means of providing medicinal treatment for chronic medical conditions. This method of medication delivery by using the gastric coil 12 according to the present invention has the advantage of requiring minimal modification of the currently available pharmaceutical agents and increasing the likelihood of patient compliance with respect to administration of medications. As shown for example in FIG. 6, the medication core 35 is placed within the interior cavity 32 of the strand 26 that is used to form the gastric coil 12.

The medication core 35 may contain medications, for example drugs and other pharmaceutical agents, which are packaged inside the gastric coil 12. The medication core 35 may be enveloped in an impermeable membrane (not shown) to prevent it from immediate and/or premature disintegration. The medication core 35 may be configured for dissolution at a predetermined rate starting at the face 38 to deliver the medication contained within the medication core 35 to the patient 150 in a continuous fashion in order to assist in the control of chronic medical conditions and to enhance the patient's 150 compliance with the administration of the medication. The medication core 35 may vary in length compared to the length of the strand 26 that forms the gastric coil 12. For example, the length of the medication core 35 may depend on the medical condition to which the medication in the medication core 35 is intended to treat and the length of time the treatment is required. It is understood that while the medication core 35 may fill the entire space of the interior cavity 32 of the strand 26, the present invention is not limited to such configurations, and it is understood that the medication core 35 may extend to only a portion of the length of the strand 26 that forms the gastric coil 12. The rate of dissolution of the medication core 35, once the gastric coil 12 containing the medication core 35 is implanted into the stomach 165 of the patient 150, will depend on the medication core's 35 pharmacologic and physical properties. The medication core 35 may be designed so that the duration of the release of medication from the medication core 35 is known so that the duration that the gastric coil 12 should be implanted into the patient 150 is known. For example, depending upon the medication condition being treated, a single gastric coil 12 may be implanted during the course of the treatment, or multiple gastric coils 12 may be implanted and removed from the patient 150 during the course of the treatment.

The gastric coil 12 may be prefabricated with the medication core 35 enclosed within the gastric coil 12. It is understood that the medication core 35 preferably should be fabricated so as to be at least as flexible and resilient as the gastric coil 12 so that the medication core 35 is not damaged, interrupted or otherwise made ineffective during the implantation of the gastric coil 12 into the stomach 165 of the patient 150 as discussed above with respect to FIG. 3. The medication core 35 may be in solid, powder, gel or any other appropriate form so as to effectively deliver medication from within the gastric coil 12 in accordance with the present invention. While it is understood that the gastric coil 12 may be used for purposes of the treatment of obesity and medication delivery, it may be preferable that the gastric coil 12 designed for medication delivery be of a size at least somewhat smaller than the size of the stomach 165 to which it is intended to be implanted into. In this manner, the gastric coil 12 may not create an unwanted sensation of fullness for the patient 150. However, it is further understood that use of the gastric device 12 in the treatment of obesity and for medication delivery are not mutually exclusive, and accordingly the same gastric device 12 may serve both purposes.

It is to be understood that all of the present figures, and the accompanying narrative discussions of corresponding embodiments, do not purport to be completely rigorous treatments of the invention under consideration. It is to be understood that the above-described arrangements are only illustrative of the application of the principles of the present invention. Numerous modifications and alternative arrangements may be devised by those skilled in the art without departing from the scope of the present invention.

What is claimed is:

1. An intragastric device, comprising:
a strand having a first end and a second end, wherein the strand is configurable between a substantially linear configuration of the intragastric device and a substantially coiled configuration of the intragastric device;
wherein in the substantially coiled configuration of the intragastric device the strand is coiled so as to produce a plurality of spaced apart loops;
wherein the plurality of spaced apart loops of the strand are positioned to define an interior region of the substantially coiled configuration of the intragastric device; and
wherein the substantially coiled configuration of the intragastric device is at least partially compressible in a first direction.

2. The intragastric device according to claim 1, wherein the strand is configured to resiliently return to the substantially coiled configuration of the intragastric device when placed in the substantially linear configuration of the intragastric device.

3. The intragastric device according to claim 1, wherein the strand comprises a longitudinal axis, and at least a portion of the strand is comprised of a helical coil positioned around the longitudinal axis of the strand, and wherein the helical coil defines a bore within the portion of the strand comprised of the helical coil.

4. The intragastric device according to claim 1, wherein in the substantially coiled configuration of the intragastric device the first end and the second end are each oriented so as to extend into the interior region of the substantially coiled configuration of the intragastric device.

5. The intragastric device according to claim 1, wherein the strand is comprised of a resilient material having shape memory.

6. The intragastric device according to claim 5, wherein the resilient material comprises a titanium alloy.

7. The intragastric device according to claim 3, wherein the bore of the helical coil comprises a cylindrical medication core disposed in at least a portion of the bore.

8. The intragastric device according to claim 1, wherein a shape of the substantially coiled configuration of the intragastric device defined by the plurality of spaced apart loops is one of spheroid, ovoid, ellipsoid, stomach or bean.

9. The intragastric device according to claim 1, wherein the intragastric device is configured in the substantially coiled configuration of the intragastric device to expand a stomach of a patient in order to produce a sense of fullness in the patient.

10. A method of treating obesity using an intragastric device, comprising:
implanting the intragastric device into a gastrointestinal tract of a patient,
wherein the intragastric device comprises a strand having a first end and a second end,
wherein the strand is configurable between a substantially linear configuration of the intragastric device and a substantially coiled configuration of the intragastric device,
wherein in the substantially coiled configuration of the intragastric device the strand is coiled so as to produce a plurality of spaced apart loops,
wherein the plurality of spaced apart loops of the strand are positioned to define an interior region of the substantially coiled configuration of the intragastric device, and
wherein the substantially coiled configuration of the intragastric device is at least partially compressible in a first direction.

11. The method according to claim 10, wherein the intragastric device is configured in the substantially coiled configuration of the intragastric device to expand a stomach of the gastrointestinal tract of the patient in order to produce a sense of fullness in the patient.

12. The method according to claim 10, wherein implanting the intragastric device into the gastrointestinal tract of the patient comprises:
configuring the strand into the substantially linear configuration of the intragastric device,
inserting the first end of the strand into an opening of the gastrointestinal tract of the patient,
moving the first end of the strand along the gastrointestinal tract of the patient towards a stomach of the gastrointestinal tract of the patient, and
inserting the second end of the strand into the stomach of the gastrointestinal tract of the patent so that the strand resiliently returns to the substantially coiled configuration of the intragastric device within the stomach.

13. The method according to claim 10, wherein the strand comprises a longitudinal axis, and at least a portion of the strand is comprised of a helical coil positioned around the longitudinal axis of the strand, and wherein the helical coil defines a bore within the portion of the strand comprised of the helical coil.

14. A method of administering at least one medication to a patient using an intragastric device, comprising:
implanting the intragastric device into a gastrointestinal tract of a patient,
wherein the intragastric device comprises a strand having a first end and a second end,
wherein the strand is configurable between a substantially linear configuration of the intragastric device and a substantially coiled configuration of the intragastric device,
wherein in the substantially coiled configuration of the intragastric device the strand is coiled so as to produce a plurality of spaced apart loops,
wherein the plurality of spaced apart loops of the strand are positioned to define an interior region of the substantially coiled configuration of the intragastric device, and
wherein the substantially coiled configuration of the intragastric device is at least partially compressible in a first direction.

15. The method according to claim 14, wherein the strand comprises a longitudinal axis, and at least a portion of the strand is comprised of a helical coil positioned around the longitudinal axis of the strand, and wherein the helical coil defines a bore within the portion of the strand comprised of the helical coil.

16. The method according to claim 15, wherein the bore of the helical coil comprises a cylindrical medication core disposed in at least a portion of the bore.

17. The method according to claim 14, wherein implanting the intragastric device into the gastrointestinal tract of the patient comprises:
configuring the strand into the substantially linear configuration of the intragastric device,
inserting the first end of the strand into an opening of the gastrointestinal tract of the patient,
moving the first end of the strand along the gastrointestinal tract of the patient towards a stomach of the gastrointestinal tract of the patient, and
inserting the second end of the strand into the stomach of the gastrointestinal tract of the patent so that the strand resiliently returns to the substantially coiled configuration of the intragastric device within the stomach.

18. The method according to claim 16, wherein the cylindrical medication core comprises the at least one medication, and is configured to release the at least one medication into the gastrointestinal tract of the patient once the intragastric device has been implanted into the gastrointestinal tract of the patient.
